(19) United States
(12) Patent Application Publication
(10) Pub. No.: US 2004/0081655 A1
(43) Pub. Date: Apr. 29, 2004

(54) Methods and compositions comprising polycationic compounds

(76) Inventors: Karen Lingnau, Vienna (AT); Alena Egyed, Vienna (AT); Walter Schmidt, Vienna (AT); Michael Buschle, Perchtoldsdorf (AT); Sonia Grill, Linz (AT)

Correspondence Address: Mark B Wilson, Fulbright & Jaworski, Suite 2400, 600 Congress Avenue, Austin, TX, 78701, US

(21) Appl. No.: 10/250,777

(22) PCT Filed: Jan. 7, 2002
(86) PCT No.: PCT/EP02/00071

(30) Foreign Application Priority Data
Jan. 5, 2001 (EP) 0100087
Apr. 25, 2001 (AT) A 670/01

(51) Int. Cl. A61K 39/00; A61K 39/12; A61K 39/02

(52) U.S. Cl. 424/185.1; 424/204.1; 424/234.1

(57) ABSTRACT

The invention relates to the use of a polycationic compound for the preparation of a medicament for treating or preventing inflammation and for the preparation of a medicament with reduced inflammatory potential.

The invention relates to new uses for polycationic compounds.

Pharmaceutically used polycationic compounds, for example the polycationic amino acid polymers poly-L-arginine and poly-L-lysine, have been shown to allow very efficient charging of antigen presenting cells (APCs) with antigens in vitro and in vivo. This is thought to be the key event for triggering immune cascades, eventually leading to the induction of antigen specific immune effector cells that are able to destroy or neutralise targets. It has been shown previously that a number of polycationic compounds excert effects on immune cells (Buschle et al., Gene Ther. Mol. Biol. 1 (1998), 309-321; Buschle et al., Proc. Natl. Acad. Sci. USA, 94 (1997), 3256-3261).

Co-injection of a mixture of poly-L-arginine and poly-L-lysine together with an appropriate antigen as a vaccine protect animals from tumor growth in several animal models. A vaccine consisting of polycationic compounds and antigens is accepted in the art as being a very effective form of treatment (WO 97/30721).

Many pharmaceutically active compounds used in the treatment or in the prevention of diseases show inflammation as side-effect, pharmaceutical application of such active substances having an inflammatory potential is often carefully weighed against the risk of inflammation induced by such a drug and severely reduces the scope of application of such drugs.

It is therefore an object of the present invention to provide means for treating or preventing inflammation, especially inflammatory side-effects. It is a further object to provide means for lowering or completely eliminating (inflammatory) side effects of medicaments.

These objects are solved by the use of a polycationic compound for the preparation of a medicament for treating or preventing inflammation. It has been surprisingly discovered that polycationic compounds may be used to prevent inflammatory effects in an individual having or being at risk of inflammation or, alternatively, to reduce the inflammatory potential of a medicament or a medical treatment significantly which allows the administration of medicaments that are usually not administered or only rarely administered due to their inflammatory side-effects.

This anti-inflammatory effect of polycationic compounds used according to the present invention may be observed both locally and systemically.

The present invention is especially beneficial if the combined medicament is administered, e.g. subcutaneously, intravenously, intranasally, intramusculary, intradermally or transdermally. However, other application forms, such as parenteral or topical application, are also suitable for the present invention. However, the depot effect seems to be mostly significant if the composition is injected or implanted.

The antigen to be used within the course of the present invention is not critical, it may preferably be selected from the group consisting of an antigen from a viral or a bacterial pathogen, an antigen from an eucaryotic pathogen, a tumor antigen, an autoimmune antigen or mixtures thereof. Especially preferred are negatively charged antigens or hydrophobic antigens. Further examples of antigens are whole-killed organisms, such as inactivated viruses or bacteria, fungi, protozoa or even cancer cells. Antigens may also consist of subfractions of these organisms/tissues, of proteins, or, in their most simple form, of peptides. Antigens can also be recognised by the immune system in form of glycosylated proteins or peptides and may also be or contain polysaccharides or lipids. Short peptides can be used, since e.g. cytotoxic T cells (CTL) recognise antigens in form of short usually 8-11 amino acids long peptides in conjunction with major histocompatibility complex (MHC). B cells recognise longer peptides starting at around 15 amino acids. By contrast to T cell epitopes, the three dimensional structure of B cell antigens may also be important for recognition by antibodies.

Preferred pathogens are selected from human immune deficiency virus (HIV), hepatitis A and B viruses, hepatitis C virus (HCV), Rous sarcoma virus (RSV), Epstein Barr virus (EBV), Influenza virus, Rotavirus, Staphylococcus aureus, Chlamydia pneumoniae, Chlamydia trachomatis, Mycobacterium tuberculosis, Streptococcus pneumoniae, Bacillus anthracis, Vibrio cholerae, Plasmodium sp. (Pl. falciparum, Pl. vivax, etc.), Aspergillus sp. or Candida albicans. Antigens may also be molecules expressed by cancer cells (tumor antigens). Antigens may also be derived antigens. The derivation process may include the purification of a specific protein from the pathogen/cancer cells, the inactivation of the pathogen as well as the proteolytic or chemical derivatisation or stabilisation of such a protein. In the same way also tumor antigens (cancer vaccines) or autoimmune antigens may be used together with a polycationic compound according to the present invention.

The polycationic compound(s) to be used according to the present invention may be any polycationic compound, which shows e.g. the characteristic effect according to the WO 97/30721, or others like cationic liposomes, polyethylene-amine, chitosan, poly cations used for DNA transfer, etc. Preferred polycationic compounds are selected from basic polypeptides, organic polycations, basic polyaminoacids or mixtures thereof. These polyaminoacids should have a chain length of at least 4 amino acid residues (see: Tuftsin as described in Goldman et al (1983)). Especially preferred are substances containing peptidic bounds, like polylysine, polyarginine and polypeptides containing more than 20%, especially more than 50% of basic amino acid residues in a range of more than 8, especially more than 20, amino acid residues or mixtures thereof. Other preferred polycations and their pharmaceutical compositons are described in WO 97/30721 (e.g. polyethyleneimine) and WO 99/38528. Preferably these polypeptides contain between 5 and 500 amino acid residues, especially between 10 and 200 residues.

These polycationic compounds may be produced chemically or recombinantly or may be derived from natural sources.

Cationic (poly)peptides may also be polycationic anti-bacterial microbial peptides with properties as reviewed in (Ganz and Lehrer, 1999; Hancock, 1999). These (poly)peptides may be of prokaryotic or animal or plant origin or may be produced chemically or recombinantly (Andreu and Rivas, 1998; Ganz and Lehrer, 1999; Simmaco et al., 1998). Peptides may also belong to the class of defensins (Ganz, 1999; Ganz and Lehrer, 1999). Sequences of such peptides can, for example, be found in the Antimicrobial Sequences Database under the following internet address:

http://www.bbcm.univ.trieste.it/˜tossi/pag1.html

Such host defense peptides or defensines are also a preferred form of the polycationic polymer according to the present invention. Generally, a compound allowing for activation (or down-regulation) of the adaptive immune system, preferably mediated by APCs (including dendritic cells) is used as polycationic polymer.

Especially preferred for use as polycationic substance in the present invention are cathelicidin derived anti-microbial peptides or derivatives thereof (A 1416/2000, incorporated herein by reference), especially anti-microbial peptides derived from mammal cathelicidin, preferably from human, bovine or mouse.

Polycationic compounds derived from natural sources include HIVREV or HIV-TAT (derived cationic peptides, antennapedia peptides, chitosan or other derivatives of chitin) or other peptides derived from these peptides or proteins by biochemical or recombinant production. Other preferred polycationic compounds are cathelin or related or derived substances from cathelin. For example, mouse cathelin is a peptide which has the amino acid sequence NH₂-RLAGLLRKGGEKIGEKLKKIGOKIKNFFQKLVPQPE-COOH. Related or derived cathelin substances contain the whole or parts of the cathelin sequence with at least 15-20 amino acid residues. Derivations may include the substitution or modification of the natural amino acids by amino acids which are not among the 20 standard amino acids. Moreover, further cationic residues may be introduced into such cathelin molecules. These cathelin molecules are preferred to be combined with the antigen and the immunogenic ODNs according to the present invention. However, these cathelin molecules surprisingly have turned out to be also effective as an adjuvant for an antigen without the addition of further adjuvants. It is therefore possible to use such cathelin molecules as efficient adjuvants in vaccine formulations with or without further immunostimulatory substances.

Another preferred polycationic substance to be used according to the present invention is a synthetic peptide containing at least 2 KLK-motifs separated by a linker of 3 to 7 hydrophobic amino acids (A 1789/2000, incorporated herein by reference).

As mentioned above polycationic compounds may according to the present invention be preferably used together with a medicament for which an inflammatory potential is known. Since the anti-inflammatory properties of polycationic compounds are according to the present invention local and systemic, the use according to the present invention may be reduced to practice by either providing a combined medicament comprising a compound with an inflammatory potential together with the polycationic compounds or by providing a medicament kit comprising a medicament with an inflammatory potential and a separated medicament comprising the polycationic compound whereby, both medicaments of the kit may be administered separately, both with respect to the administration time and the administration site. However, preferably the compounds are administered at the same site and time. Also the form, in which the combined medicament between the poly-cations and the compounds to be applied is administered is not critical, the combined medicaments may therefore be e.g. a mixture or covalently coupled.

Preferred compounds with inflammatory potential to be used within the course of the present invention are immunogenic nucleic acid molecules. It is known that the immune system of mammals (and probably most if not all vertebrates) recognises DNA of lower organisms, including bacteria probably due to structural and sequence usage differences between pathogen and host DNA. In particular, short stretches of DNA derived from neon-vertebrates or short form of oligodeoxynucleotides (ODNs) containing non-methylated cytosine-guanine dinucleotides (CpG) in a certain base context, are targeted. CpG motifs are found at the expected frequency in bacterial DNA but are much less frequent in vertebrate DNA. In addition, non-vertebrate (i.e. bacterial) CpG motifs are not methylated, whereas vertebrate CpG sequences are. Such ODNs containing CpG motifs (CpG-ODNs) can directly activate monocytes and B cells. In consequence, the activation of monocytes and NK cells by CpG-ODNs promotes the induction of a Th1-type response and the development of cytotoxic T cells. In addition, such immunogenic ODNs are used as vaccine adjuvants to enhance the antibody response to specific antigens (e.g. EP 0 468 520 A2, WO 96/02555, WO 98/16247, etc.). However, these CpG-ODNs exhibit strong inflammatory potential, an administration of CpG-ODNs is connected with severe local and systemic inflammatory events.

Since CpG-ODNs show local as well as systemic inflammatory reactions, but at the same time have also a potential to be used as a beneficial stimulatory medicament, this substance was applied to an animal to create and provide a model to show the advantages of the present invention. Indeed, it could be shown that both, the local and the systemic inflammatory events caused by administration of CpG-ODNs may be completely inhibited by administration of polycationic compounds.

Therefore, a preferred embodiment of the present invention is characterised in that the medicament is to be applied together with the polycationic compound further comprises immunogenic oligodesoxy nucleic acid molecules (ODNs), especially ODNs containing CpG motifs (CpG-ODNs), inosine containing ODNs (1-ODNs) or mixtures or combinations thereof. I-ODNs are described for example in the Austrian patent application A 1973/2000 (incorporated herein by reference). Mixtures of I-ODNs with CpG-ODNs may also be provided as well as combinations of these two principles, e.g. an I-ODN containing CpG motifs.

It is known (PCT/EP 01/00087) that the co-application of polycationic compounds and CpG-ODNs with an antigen strongly and synergystically enhances the induction of an antigen specific immune response when compared to the injection without poly-cationic compounds. That is reflected by a high number of IFN-γ-producing cells isolated from draining lymph nodes (ELISPOT assay). As stated above within the course of the present invention it could be shown that this strong local immune response (day 4/draining lymph node cells) induced after one single injection of an antigen with a mixture of polycationic compounds (as an example polyarginine pR 60 is used) and CpG-ODNs converts to a systemic immune response which is very long lasting. According to the present invention, the complex formation ability of substances such as CpG-ODNs with polycationic compounds is used for preventing a systemic distribution and the subsequent fast resorption of such substances, thereby providing a strong prolongation of the properties of such substances, e.g. a prolongation of the immunostimulatory properties of CpG-ODNs. In addition, preventing the systemic distribution avoids the induction of potential harmful systemic side effects of immunostimulatory agents.

This model using CpG-ODNs and polycationic peptides is further described and analysed in the example section. Moreover, to provide an analysable pharmaceutical target, an Ovalbumin-derived peptide (OVA₂₅₇₋₂₆₄) is used as a model compound (a model antigen).

A second preferred compound with inflammatory potential to be used within the course of the present invention is lipopolysaccharide. (LPS, endotoxin), a cell wall component of Gram-negative bacteria. LPS plays a pivotal role in the induction of septic shock (endotoxic shock) (Karima et al (1999). In particular, circulatory failure, leukocyte-induced tissue injury and activation of coagulation systems appear to be critical determinants in the development of sequential organ failure. Furthermore, lethal shock can be associated with excessive secretion of pro-inflammatory cytokines like TNF-a and IL-6.

The model using LPS in combination with polycationic peptides is further described and analysed in the example section.

The present invention also relates to a method for treating or preventing inflammation, comprising administering to an individual (e.g. Patient or animal), suffering from inflammation or being at risk thereto, an effective amount of a polycationic compound. The invention further relates to a method for reducing the inflammatory potential of a medicament when administering said medicament to a patient, comprising administering that medicament together with an effective amount of a polycationic compound.

The amounts of polycationic compound to be administered is highly depending on the necessities of the individual composition and optionally on the drug to be administered together with the polycationic polymer. In case of poly-L-arginine and poly-L-lysine preferred amounts of polycation are 0.001-1000 μg/administration unit, more preferred 0.1-10 mg/dose, especially around or beyond 0.1 mg/20 g body weight (of mice) or the equivalent dose for humans.

The invention will be described in more detail by way of the following examples and the drawing figures, yet it is not restricted to these particular embodiments.

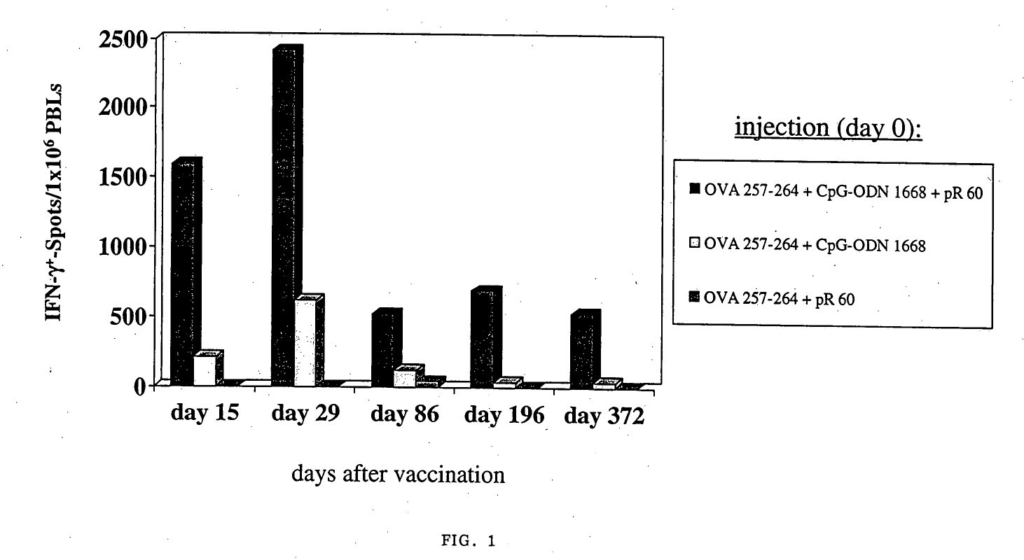

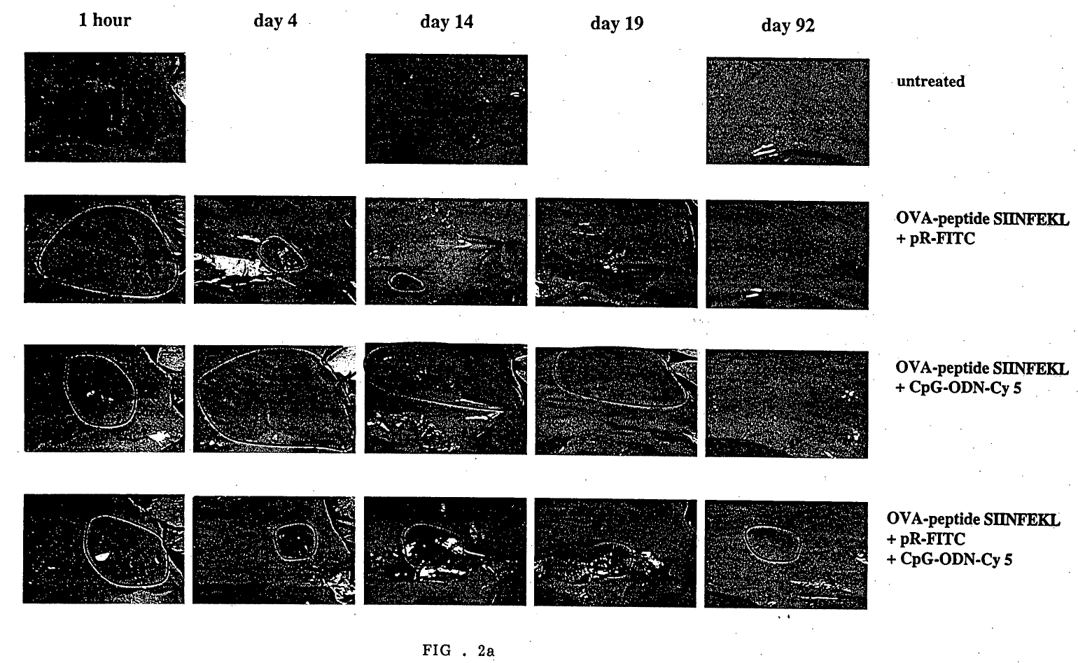

FIG. 1 shows that the combined application of poly-L-arginine, CpG-ODN and antigen induces strong antigen-specific immune responses which are systemic and very long lasting. The figure shows peripheral blood lymphocytes stimulated ex vivo with OVA₂₅₇₋₂₆₄-peptide;

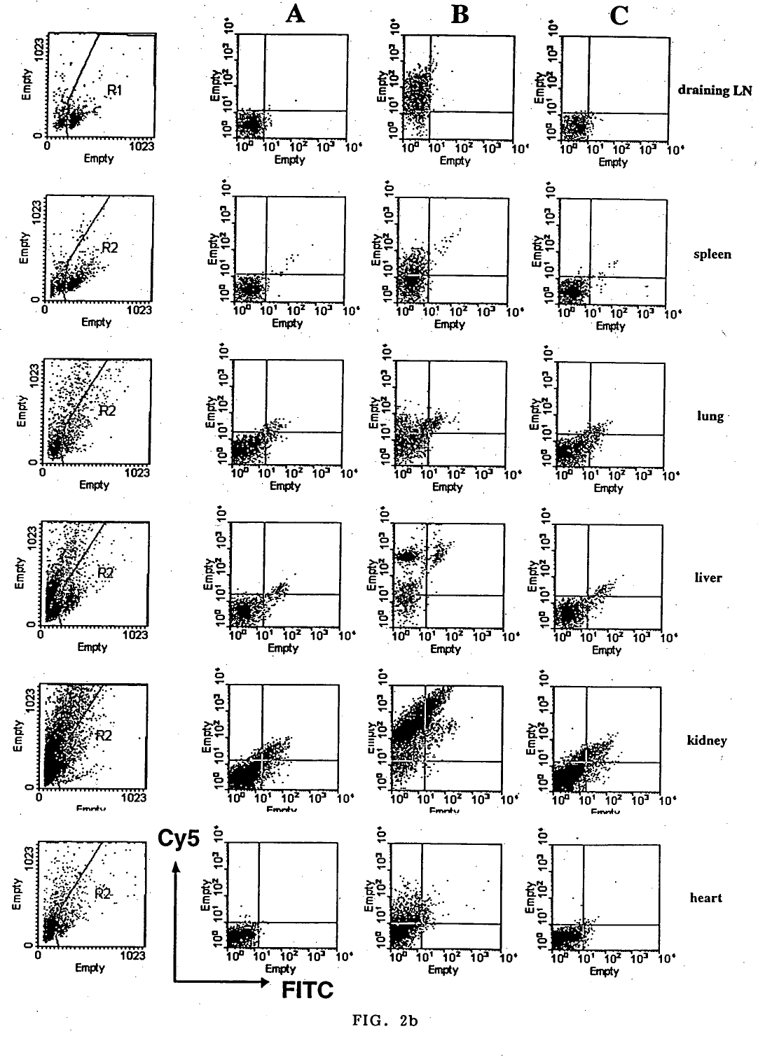

FIG. 2a shows that poly-L-arginine induces the formation of a depot at the injection site. This figure shows photos from the injection sites at the indicated time points after vaccination. White lines surround the area where the fluorescence labelled compounds of the vaccine can be detected;

FIG. 2b shows that the co-application of poly-L-arginine inhibits the spreading of CpG-ODN-Cy5 throughout the body. This figure shows FACS analyses of lymphoid and non-lymphoid tissues at day 1 after injection of CpG-ODN-Cy5 (B) or CpG-ODN-Cy5 and pR 60-FITC (C). Untreated mice were used as a control (A);

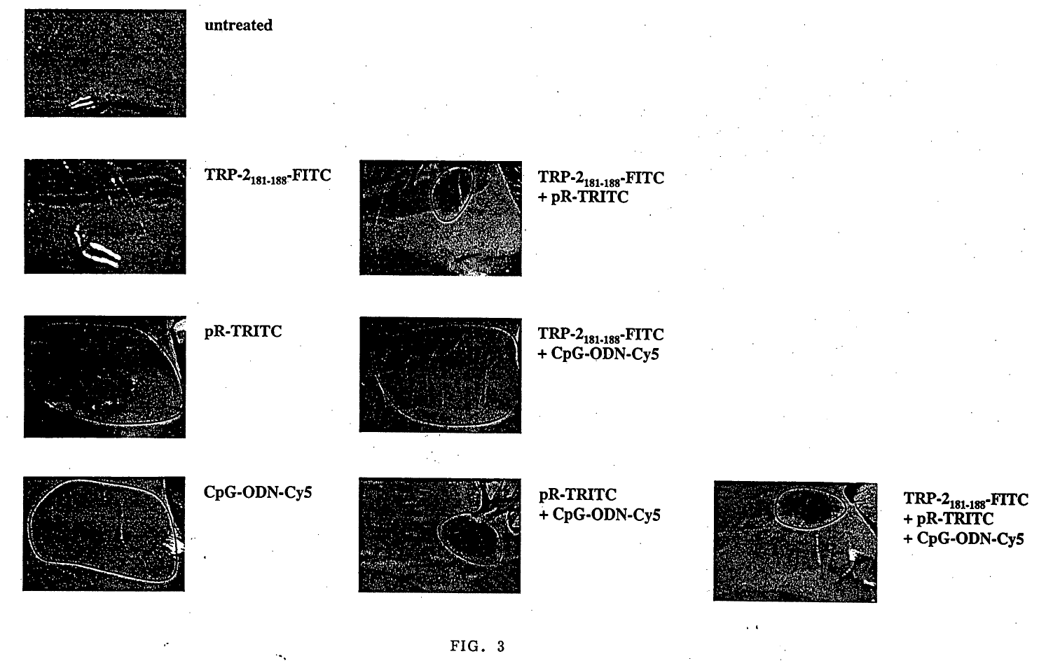

FIG. 3 shows that poly-L-arginine induces the formation of a depot at the injection site when co-injected at least with one more molecule. This figure shows photos from the injection sites at day 4 after vaccination;

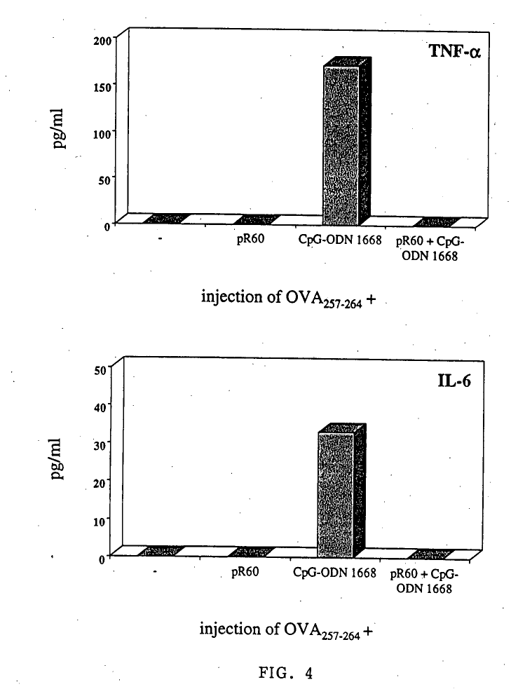

FIG. 4 shows that co-injected poly-L-arginine prevents the CpGODN-induced systemic production of TNF-α and IL-6 in vivo. Mice were injected into the hind footpads and one hour later serum was prepared. The amount of TNF-α and IL-6 in the sera was determined by ELISA;

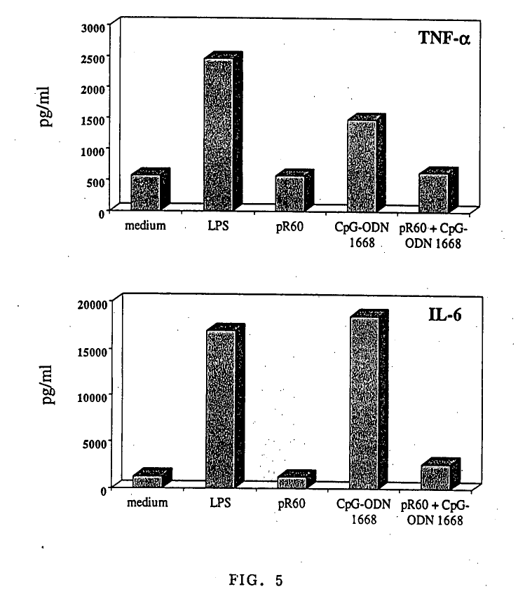

FIG. 5 shows that poly-L-arginine abrogates CpG-ODN-induced production of TNF-α and IL-6 by mouse BM-DC in vitro. CD11c⁺ sorted BM-DC were incubated either with pR60, CPG-ODN 1668 or pR60 and CpG-ODN 1668 or, for control purposes, with medium or LPS. After the incubation for 24 h, the amount of TNF-α and IL-6 were determined in the supernatant by ELISA;

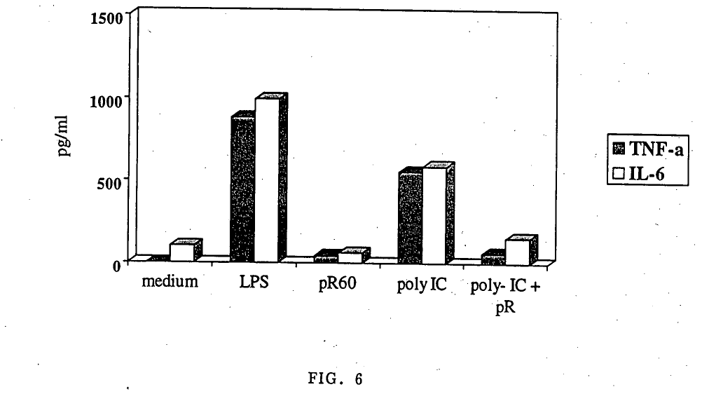

FIG. 6 shows that poly-L-arginine abrogates poly I:C-induced production of pro-inflammatory cytokines by human DC in vitro. Day 5-cultured human monocyte-derived DC were incubated (1×10⁶/well) either with poly I:C, pR60, poly I:C and pR60 or, for control purposes, with LPS and medium alone in 24-well culture plates for 24 h. Thereafter, supernatants were collected and stored at −20° C. until use. The amount of TNF-α and IL-6 in the supernatants was determined by ELISA.

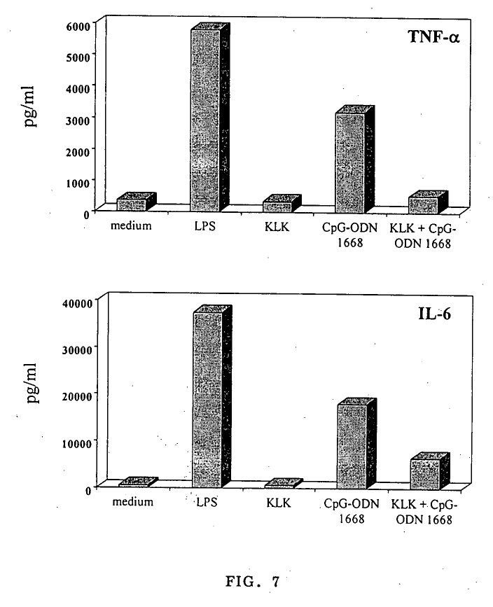

FIG. 7 shows that KLK abrogates CpG-ODN-induced production of TNF-α and IL-6 by BM-DC in vitro: CD11c⁺ sorted BM-DC were incubated either with KLK, CPG-ODN 1668 or KLK and CpG-ODN 1668 or, for control purposes, with medium or LPS. After the incubation for 24 h, the amount of TNF-α and IL-6 in the supernatants were determined by ELISA.

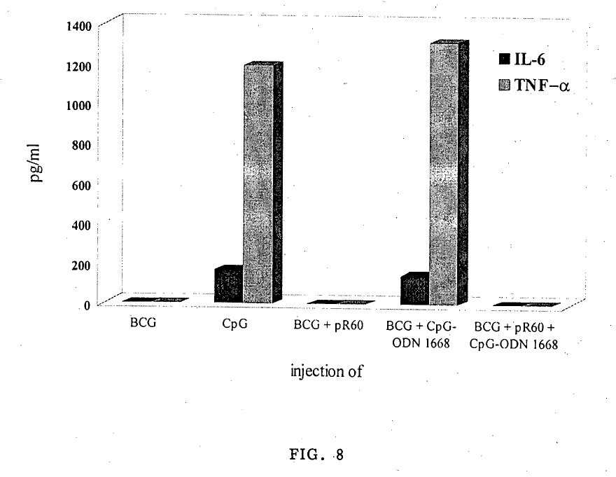

FIG. 8 shows that co-injected poly-L-arginine prevents the BCG/CpG-ODN-induced systemic production of TNF-α and IL-6 in vivo. Mice were injected s.c. Into the flank with 5×10³ cells of BCG and one hour later serum was prepared. The amount of TNF-α and IL-6 in the sera was determined by ELISA.

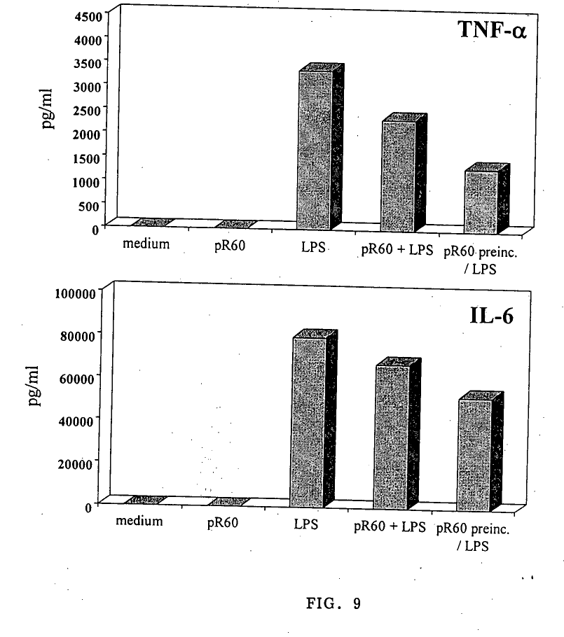

FIG. 9 shows that poly-L-arginine decrease LPS-induced production of pro-inflammatory cytokines by murine bone-marrow-derived dendritic cells in vitro.

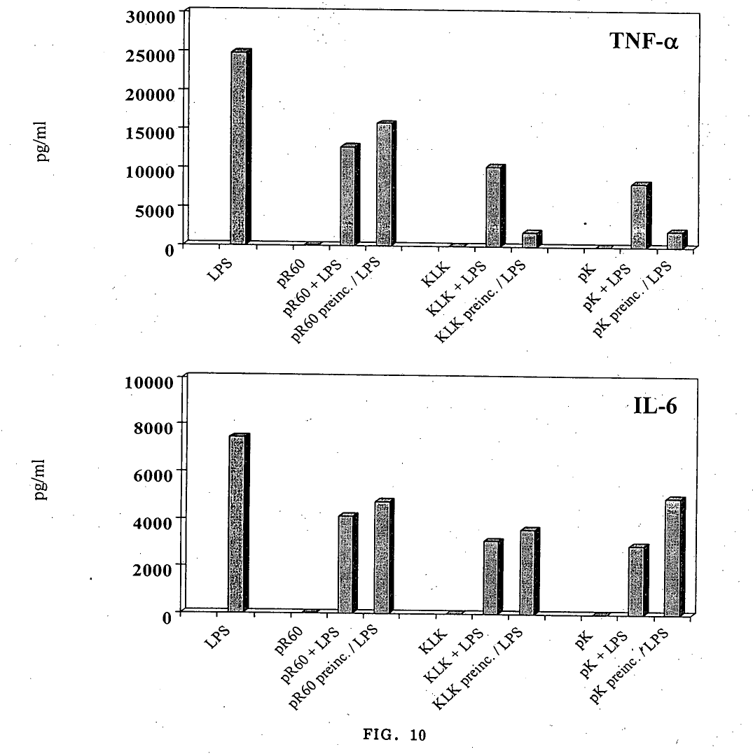

FIG. 10 shows that poly-L-arginine, KLK and poly-D-lysine decrease LPS-induced production of pro-inflammatory cytokines by human dendritic cells in vitro.

### EXAMPLES

In the present examples it is shown that the strong local immune response (day 4/draining lymph node cells) induced after one single injection of antigen with a mixture of pR60 and CpG-ODN converts to a systemic immune response which is, most importantly, very long lasting (Example 1). Even 372 days after injection (the latest time point analysed), around 500 antigen-specific, IFN-γ producing T cells per million peripheral blood lymphocytes can be detected. One possible explanation for this effect: might be that a complex-formation of CPG-ODN with poly-L-arginine prevents the systemic distribution of CpG-ODN and the subsequent fast resorption of CpG-ODN. Hence, this results in a strong prolongation of the immunostimulatory properties of CpG-ODNs.

In order to investigate this assumption, fluorescence-labeled compounds were injected together subcutaneously into the flank of mice. At different time points after this treatment, injection sites were inspected for the presence of labeled compounds. In example 2a and 2b, OVA₂₅₇₋₂₆₄-peptide (unlabeled), poly-L-arginine-FITC (yellow) and CpG-ODN-Cy5 (blue) were used for injections. After injection of OVA₂₅₇₋₂₆₄-peptide with poly-L-arginine-FITC the formation of a depot could be detected at the-injection site. The injection of OVA₂₅₇₋₂₆₄-peptide with CpG-ODN-Cy5 resulted in the distribution of CpG-ODN-Cy5 all over the skin (example 2a). As simultaneously determined by FACS analyses (example 2b), CpG-ODN-Cy5 is also detectable in secondary lymphoid organs (draining lymph node, spleen) and non-lymphoid tissues (lung, liver, kidney, heart). In contrast, when OVA₂₅₇₋₂₆₄-peptide and CpG-ODN-Cy5 were injected together with poly-L-arginine-FITC, the CpG-ODN-Cy5 was restricted to the depot formed by poly-L-arginine (example 2a). FACS analyses from these mice (example 0.2b) revealed that CpG-ODN-Cy5 is not detectable in the periphery, due to the fact that CpG-ODN-Cy5 is trapped by poly-L-arginine in the depot at the injection site. Both, poly-L-arginine-FITC and CpG-ODN-Cy5 can be detected within this depot at least up to day 92 after injection (the latest time point analysed). This observation implies that the combination of peptide and poly-L-arginine with CpG-ODN led to a far long lasting existence of the depot compared to the combination of peptide and poly-L-arginine. In example 3, TRP-2₁₈₁₋₁₈₈-peptide-FITC (yellow), poly-L-arginine-TRITC (red-violet), CpG-ODN-Cy5 (blue) were used for injections. When TRP-2₁₈₁₋₁₈₈-peptide-FITC was injected either alone or in combination with CpG-ODN-Cy5, the peptide was not detectable at the injection site at day 4. The injection of poly-L-arginine-TRITC alone resulted in its distribution all over the skin. The injection of CpG-ODN-Cy5 either alone or in combination with TRP-2₁₈₁₋₁₈₈-peptide-FITC resulted in the distribution of CpG-ODN-Cy5 all over the skin.

Thus, these findings imply that poly-L-arginine induces a depot at the injection site within other compounds (antigen and/or immunostimulatory CpG-ODN) are kept. In the case of co-injection of OVA₂₅₇₋₂₆₄-peptide, poly-L-arginine and CpG-ODN, the slow release of both peptide and CpG-ODN from this depot is most likely responsible for the persistent activation of accessory cells and subsequently the persistent stimulation of T cells. In consequence, this leads to the observed long lasting existence of high numbers of antigen-specific T cells in the periphery after one single injection.

Beside their potent immunostimulatory effects, CpG-ODNs are described to have potentially harmful side effects in that they induce the systemic release of high amounts of pro-inflammatory cytokines such as TNF-α and IL-6, which could induce a shock syndrome (Sparwasser 1997, Lipford 1997). As described in example 2a, 2b and 3, CpG-ODNs are not systemically present when injected in combination with poly-L-arginine. Therefore, it was investigated whether the co-administration of poly-L-arginine affects the CpG-ODN-induced systemic production of TNF-α and IL-6. Serum levels of both cytokines were determined by ELISA one hour after injection. Example 4 demonstrates that neither the injection of OVA₂₅₇₋₂₆₄-peptide alone nor in combination with poly-L-arginine led to the induction of significant amounts of TNF-α and IL-6 in the serum, whereas the injection of OVA₂₅₇₋₂₆₄-peptide in combination with CPG-ODN induces high concentrations of both cytokines. However, upon co-administration of OVA₂₅₇₋₂₆₄-peptide with poly-L-arginine and CpG-ODN, this systemic production of TNF-α and IL-6 was totally abolished. Thus, these data in combination with the findings demonstrated in Example 2 and 3 indicate that the localisation of CpG-ODN via the depot formation mediated by poly-L-arginine prevents the systemic distribution of CpG-ODN and subsequently the systemic release of pro-inflammatory cytokines. In parallel, in vitro studies were performed to clarify whether the complexation of CpG-ODN by poly-L-arginine can also directly influence the stimulation of mouse bone marrow-derived CD11c⁺ dendritic cells by CpG-ODN concerning the production of TNF-α and IL-6. For this purpose, CD11c³⁰ dendritic cells were incubated either with poly-L-arginine, CpG-ODN or the combination of poly-L-arginine and CpG-ODN (example 5). The levels of TNF-α and IL-6 were determined in the supernatants derived from these cultures. After incubation with poly-L-arginine neither TNF-α nor IL-6 were detectable, whereas after incubation with CpG-ODN significant amounts of both cytokines are produced. Impressively, the presence of poly-L-arginine inhibited the CpG-ODN-induced production of TNF-α and IL-6 by these cells.

Thus, these results indicate that the complexation of CpG-ODN by poly-L-arginine not only inhibits the systemic but also the local release of pro-inflammatory cytokines. In consequence, these beneficial effects of poly-L-arginine prevent probably uncontrolled and excessive systemic and local immune responses induced by CpG-ODNs.

Further in vitro-experiments revealed that poly-L-arginine also inhibits the polyinosinic-polycytidylic acid-induced production of pro-inflammatory cytokines by human dendritic cells (Example 6).

Thus, these observations imply a general anti-inflammatory effect of poly-L-arginine. The risks of the application of immunogenic but potential harmful substances can be probably minimised by the co-application of poly-L-arginine. The rapid systemic distribution of such substances can be prevented by the property of poly-L-arginine to form a depot in which all compounds are trapped. Furthermore, the complexation of these substances by poly-L-arginine can e.g. inhibit the local release of toxic amounts of pro-inflammatory cytokines.

### Example 1

The Combined Application of Ovalbumin-Peptide/Poly-L-Arginine (pR 60)/CPG-ODN Leads to the Induction of Strong Antigen-Specific Immune Responses Which are Systemic and Very Long Lasting.

Mice C57Bl/6 (Harlan/Olac)

Peptide OVA₂₅₇₋₂₆₄-Peptide (SIINFEKL), a MHC class I (H-2 Kb)-restricted epitope of chicken Ovalbumin (Rotzschke, O. et al., Eur. J. Immunol. 199121 (11): 2891-4), synthesised by standard solid phase F-moc synthesis, HPLC purified and ana lysed by mass spectroscopy for purity.

Dose: 300 μg/mouse

Poly-L-Arginine 60 (pR60)

Poly-L-Arginine with an average degree of polymerisation of 60 arginine residues; SIGMA chemi cals

Dose: 100 μg/mouse

CPG-ODN 1668 phosphothioate-modified oligodinucleotides con taining a CpG-motif: tcc atg acg ttc ctg atg ct, synthesised by NAPS Göttingen GmbH.

Dose: 5 nmol/mouse

Experimental Groups (5 Mice Per Group)

1. OVA₂₅₇₋₂₆₄-Peptide+CpG-ODN+pR 60

2. OVA₂₅₇₋₂₆₄-Peptide+CpG-ODN

3. OVA₂₅₇₋₂₆₄-Peptide+pR 60

On day 0, mice were injected into each hind footpad with a total volume of 100 μl (50 μl per footpad) containing the above mentioned compounds. Blood was taken via the tail vein at the indicated time points and peripheral blood lymphocytes (PBLs) were isolated using a Ficoll gradient. PBLs were stimulated ex vivo with the antigen used for vaccination, with medium (background) and Concanavalin A (positive control). IFN-γ-ELISPOTs were carried out as described (Miyahira et al., 1995). This method is a widely used procedure allowing the quantification of antigen-specific T cells. Spots representing single IFN-γ producing T cells were counted and the number of background spots was substracted from all samples. There were many spots detected after the stimulation with Con A (data not shown) indicating a good condition of the used lymphocytes. For each experimental group of mice the number of spots/1×10⁶ PBLs are illustrated in FIG. 1.

### Example 2a

Poly-L-Arginin Induces the Formation of a Depot at the Injection Site

Mice C57Bl/6 (Harlan/Olac)

Peptide OVA₂₅₇₋₂₆₄-Peptide (SIINFEKL), a MHC class I (H-2 Kb)-restricted epitope of chicken Ovalbumin (Rotzschke, O. et al., Eur. J. Immunol. 1991 21(11): 2891-4), synthesised by standard solid phase F-moc synthesis, HPLC purified and analysed by mass spectroscopy for purity.

Dose: 300 μg/mouse

Poly-L-Arginine 60-FITC (pR60-FITC)

Poly-L-Arginine with an average degree of polymerisation of 60 arginine residues; SIGMA chemicals

For fluorescein (FITC) labeling of poly-L-arginine, the poly-L-arginine was dissolved in 50 mM HEPES pH 7.9 (10 mg/500 μl). A 5-fold molar excess of FITC (Molecular Probes, Eugene, Oreg.) in an equal volume of DMSO was added to the poly-L-arginine solution. The solution was kept at room temperature in the dark for 2.5 hours. Unreacted dye was separated by running the mixture over a PD10 column (Pharmacia, Uppsala, Sweden), using 50 mM Hepes, pH 7.9, as eluent. The solution was then dialysed against 2×5 liter aqua dest., pH 7.4(0.1M HCL), over night. After lyophilisation poly-L-arginine FITC was dissolved in aqua bidest with a concentration of 10 mg/ml.

Dose: 100 μg/mouse

CPG-ODN 1668-Cy5

phosphothioate-modified, Cy5-labeled oligodinucleotides containing a CpG motif:

tcc atg acg ttc ctg atg ct, synthesised by NAPS Göttingen GmbH.

Dose: 5 nmol/mouse

Experimental Groups (1 Mouse/Group 1/Indicated Time Point, 3 Mice/Group 2-4/Time Point)

1. untreated

2. OVA₂₅₇₋₂₆₄-Peptide+pR 60-FITC

3. OVA₂₅₇₋₂₆₄-Peptide+CpG-ODN1668-Cy5

4. OVA₂₅₇₋₂₆₄-Peptide+pR 60-FITC+CpG-ODN 1.668-Cy5

On day 0 mice were injected subcutaneously into the right flank with a total volume of 100 μl containing the above mentioned compounds. Animals were sacrificed at the indicated time points after injection and photos were taken from the injection sites (FIG. 2a).

### Example 2b

Co-Application of Poly-L-Arginine Inhibits the Distribution of CpG-ODN-Cy5 Throughout the Body

Mice C57B1/6 (Harlan/Olac)

Poly-L-Arginine 60-FITC (pR60-FITC)

Poly-L-Arginine with an average degree of polymerisation of 60 arginine residues; SIGMA chemicals

For fluorescein (FITC) labeling of poly-L-arginine, the poly-L-arginine was dissolved in 50 mM HEPES pH 7.9 (10 mg/500 μl). A 5-fold molar excess of FITC (Molecular Probes, Eugene, Oreg.) in an equal volume of DMSO was added to the poly-L-arginine solution. The solution was kept at room tempera ture in the dark for 2.5 hours. Unreacted dye was separated by running the mixture over a PD10 column (Pharmacia, Uppsala, Sweden) using 50 mM He pes pH 7.9 as eluent. The solution was then dialysed against 2×5 liter aqua dest., pH 7.4 (0.1M HCL), over night. After lyophilisation poly-L-argine-FITC was dissolved in aqua bidest with a concentration of 10 mg/ml.

Dose: 100 μg/mouse

CPG-ODN 1668-Cy5

phophothioate-modified, Cy5-labeled oligodinucleotides containing a CpG motif: tcc atg acg ttc ctg atg ct, synthesised by NAPS Göttingen GmbH.

Dose: 5 nmol/mouse

Experimental Groups (1 Mouse/Group 1/Indicated Time Point, 3 Mice/Group 2-4/Time Point)

1. untreated

2. CpG-ODN1668-Cy5

3. pR 60-FITC+CpG-ODN 1668-Cy5

Mice were injected subcutaneously into the right flank with a total volume of 100 μl containing the above mentioned compounds. One day after injection, mice were sacrificed and FACS-analyses were performed from secondary lymphoid organs (draining lymph node, spleen) as well as non-lymphoid tissues (lung, liver, kidney, heart) (FIG. 2b).

### Example 3

Poly-L-Arginine Induces the Formation of a Depot at the Injection Site When Co-Injected at Least with One More Molecule

Mice C57Bl/6 (Harlan/Olac)

Peptide TRP-2-Peptide (VYDFFVWL), a MHC class I (H-2 Kb) restricted epitope of mouse tyrosinase related protein-2 (Bloom, M. B. et al., J Exp Med 1997 185, 453-459), synthesised by standard solid phase F-moc synthesis, HPLC purified and analysed by mass spectroscopy for purity. For fluorescein (FITC) labeling, the TRP-2₁₈₁₋₁₈₈-peptide was dissolved in 1M sodium borate, pH 7.9. An 8-fold molar excess of FITC (Molecular Probes, Eugene, Oreg.) in an equal volume of DMF was added to the peptide solution. The solution was kept at room temperature for four hours. Unreacted dye was seoarated ba running the mixture over a G25 gel filtration column (Pharmacia, Uppsala, Sweden) using 0.1% TFA in water as eluent. Two moles of FITC were incorporated per mol of peptide (N-terminus, side chain of lysine)

Dose: 100 μg/mouse

Poly-L-Arginine 60-TRITC (pR60-TRITC)

Poly-L-Arginine with an average degree of polymerisation of 60 arginine residues; SIGMA chemicals. For TRITC-labeling of poly-L-arginine, the poly-L-arginine was dissolved in 50 mM HEPES pH 7.9 (10 mg/50011). A 5-fold molar excess of FITC (Molecular Probes, Eugene, Oreg.) in an equal volume of DMSO was added to the poly-L-arginine solution. The solution was kept at room temperature in the dark for 2.5 hours. Unreacted dye was separated by running the mixture over a PD10 column (Pharmacia, Uppsala, Sweden), using 50 mM Hepes, pH 7.9, as eluent. The solution was then dialysed against 2×5 liter aqua dest., pH 7.4 (0.1M HCL), over night. After lyophilisation poly-L-argine-TRITC was dissolved in aqua bidest with a concentration of 10 mg/ml.

Dose: 100 μg/mouse

CpG-ODN 1668-Cy5

phosphothioate-modified, Cy5-labeled oligodinu cleotides containing a CpG motif: tcc atg acg ttc ctg atg ct, synthesised by NAPS Göttingen GmbH.

Dose: 5 nmol/mouse

Experimental Groups (1 Mouse/Group 1/Indicated Time Point, 3 Mice/Group 2-4/Time Point)

1. untreated

2. TRP-2₁₈₁₋₁₈₈-FITC

3. pR60-TRITC

4. CpG-ODN1668-Cy5

5. TRP-2₁₈₁₋₁₈₈-FITC+pR60-TRITC

6. TRP-2₁₈₁₋₁₈₈-FITC+CpG-ODN-Cy5

7. pR60-TRITC+CpG-ODN 1668-Cy5

8. TRP-2₁₈₁₋₁₈₈-FITC+pR 60-TRITC+CpG-ODN 1668-Cy5

On day 0 mice were injected subcutaneously into the right flank with a total volume of 10041 containing the above mentioned compounds. Animals were sacrificed at day 4 after injection and photos were taken from the injection sites (FIG. 3).

### Example 4

The Co-Injection of Poly-L-Arginine Prevents the CpG-ODN-Induced Systemic Production of TNF-α and IL-6 In Vivo

Mice C57Bl/6 (Harlan/Olac)

Peptide OVA₂₅₇₋₂₆₄ (SIINFEKL), an MHC class I (H-2 Kb)-restricted epitope of chicken ovalbumin (Rotzschke et al., 1991), was synthesised using standard solid phase F-moc synthesis, HPLC-purified and analysed by mass spectroscopy for purity

Dose: 300 μg/mouse

Poly-L-arginine 60 (pR60)

Poly-L-arginine with an average degree of polymerisation of 60 arginine residues; SIGMA Chemicals

Dose: 100 μg/mouse

CpG-ODN 1668 phosphothioate-modified oligodeoxynucleotides containing a CpG motif: TCC ATG ACG TTC CTG ATG CT, synthesised by NAPS GmbH, Göttingen.

Dose: 5 nmol/mouse

Experimental Groups: 4 Mice Per Group

1. OVA₂₅₇₋₂₆₄

2. pR60

3. CPG 1668+OVA₂₅₇₋₂₆₄

4. CpG 1668+pR60+OVA₂₅₇₋₂₆₄

Mice were injected into each hind footpad with a total volume of 100 μl (50 μl per footpad), containing the above mentioned compounds. One hour after injection blood was taken from the tail-vein and serum was prepared. The amount of the pro-inflammatory cytokines TNF-α and IL-6 in the sera was determined by cytokine-specific ELISAs according to the manufacturer's instructions (R&D Systems, Inc., Minneapolis, Minn.).

This experiment shows that injection of OVA₂₅₇₋₂₆₄ alone or in combination with poly-L-arginine does not-induce the production of detectable amounts of TNF-α or IL-6 (FIG. 4). In contrast, the injection of OVA₂₅₇₋₂₆₄-peptide with CpG-ODN 1668 induces the systemic production of TNF-α and IL-6. When peptide and CpG-ODN were co-injected with poly-L-arginine, the CpG-ODN induced production of pro-inflammatory cytokines was inhibited.

### Example 5

Poly-L-Arginine Abrogates CpG-ODN-Induced Production of Pro-Inflammatory Cytokines by Murine Bone Marrow-Derived Dendritic Cells In Vitro

Lipopolysaccharide (L.PS)

Lipopolysaccharide from Escherichia coli; serotype 0111:B4 (SIGMA Chemicals)

Dose: 100 ng/ml

Poly-L-arginine 60 (pR60)

Poly-L-arginine with an average degree of polymerisation of 60 arginine residues; SIGMA Chemicals, P-4663, Lot 68H5903

Dose: 10 μg/ml

CPG-ODN 1668 phosphothioate-modified oligodeoxynucleotides containing a CpG-motif: tcc atg acg ttc ctg atg ct, synthesised by NAPS Göttingen GmbH.

Dose: 1 nmol/ml

Experimental Groups

1. culture medium

2. LPS

3. pR

4. CpG-ODN 1668

5. pR+CpG-ODN 1668

Murine dendritic cells (DC) were generated from bone-marrow precursor cells of C57Bl/6 mice as previously described (Inaba 1992). Briefly, bone marrow cells were obtained by flushing the femurs and tibias with medium. Cells were depleted of lymphocytes, granulocytes and MHC class II⁺cells by Ab-complement-mediated lysis. Dead cells were removed by Ficoll gradient (Ficoll Separating Solution, density 1.077 g/mol; Biochrom KG, Germany). The remaining cells (1×10⁶/ml) were cultured in 24-well culture plates in high glucose DMEM medium (PAA Laboratories GmbH, Linz, Austria; E 15-009) supplemented with 10% heat-inactivated FCS, 10. mM sodium pyruvate, 2 mM L-glutamine, 50 mg/ml gentamicin, 0.5 mM 2-mercaptoethanol (all supplements from PAA Laboratories GmbH, Linz, Austria), 20 ng/ml GM-CSF and 250 U/ml IL-4. On day 3 of culture, cells were fed with fresh medium. On day 6 of culture, non-adherent cells and loosely adherent aggregates were harvested, washed and re-plated in 6-well culture plates (2×10^{6/5} ml). After additional 1 day of culture, non-adherent cells were collected for analyses. Using this protocol, we routinely obtained 50-60% MHC class II⁺/N418+cells as revealed by flow cytometryl. For in vitro-stimulations, day 7-cultured bone marrow cells were further enriched for CD11c⁺ cells (=DC) using magnetic beads (Miltenyi, Bergisch-Gladbach, Germany). The purity of BM-DC was more than 95% as determined by flow cytometry analyses. CD11c⁺ sorted BM-DC were incubated either with poly-L-arginine (10 μg/ml), CpG-ODN 1668 (1 nmol/ml) or the combination of poly-L-arginine and CPG-ODN 1668, and, for control purposes, with medium or LPS. Supernatants were harvested after 24 h and analysed for the production of TNF-α and IL-6 using specific ELISAs.

This experiment revealed that CPG-ODN 1668 induces the production of TNF-α and IL-6 by murine BM-DC (FIG. 5). When murine BM-DC were stimulated with poly-L-arginine alone, we could not detect any of the mentioned pro-inflammatory cytokines in DC-derived supernatants. The CpG-ODN 1668-induced production of TNF-α and IL-6 was inhibited when BM-DCs were stimulated with a mixture of CpGODN 1668 and poly-L-arginine.

### Example 6

Poly-L-Arginine Abrogates Poly I:C-Induced Production of Pro-Inflammatory Cytokines by Human DC.

Lipopolysaccharide (LPS)

Lipopolysaccharide from Escherichia coli; serotype 0111:B4 (SIGMA Chemicals)

Dose: 10 ng/ml

Poly-L-arginine 60 (pR60)

Poly-L-arginine with an average degree of polymerisation of 60 arginine residues; SIGMA Chemicals, P-4663, Lot 68H5903

Dose: 10 μg/ml

Polyinosinic-polycytidylic acid (pIC)

Polyinosinic-polycytidylic acid (Amersham Pharmacia Biotech, 27-4732, Lot 6034732012).

Dose: 10 μg/ml

Experimental Groups

1. culture medium

2. LPS

3. pR

4. pIC

5. pR+pIC

Human DC were generated from monocytes. Briefly, peripheral blood leukocytes (PBLs) were isolated from buffy coats of healthy volunteers by Ficoll gradient centrifugation. Monocytes were isolated from PBLs using CD14-coated magnetic beads (Miltenyi Biotec Inc., Germany) applied according to the manufacturer's instructions. Using this method, we obtained >95% CD14⁺ cells as determined by flow cytometry. These CD14⁺ monocytes were cultured in RPMI 1640 medium supplemented with 10% FCS (PAA Laboratories, Linz, Austria), non-essential aminoacids, L-glutamin, gentamicin, sodium pyruvate, 100 ng/ml human GM-CSF and 500 U/ml human IL-4 in 6-well tissue plates for 6-7 days. To this end, the cultures contained >80%. MHC class II⁺/CD1a⁺ cells (=DC).

To determine, which cytokines are induced/produced by DC upon poly I:C and/or pR stimulation, DC were incubated with different stimuli for 12 and 24 hours, supernatants were harvested, stored at −20° C. and screened for the presence of respective cytokines by specific ELISAs. These experiments revealed that poly I:C triggers the production of TNF-α and IL-6 in human DC (FIG. 6). In addition, human DC produced IL-12/p70 upon poly I:C treatment, which is in accordance with published data (Verdijk 1999). When human DC were stimulated with pR alone, we could not detect any of the above mentioned cytokines in DC-derived supernatants. Interestingly, poly I:C-induced TNF-α and IL-6 production was inhibited when DC were stimulated with a mixture of pIC and pR. Comparable results were obtained when DC were stimulated for 12 and 24 hours.

These results indicate that poly I:C activates human DC to produce pro-inflammatory cytokines in vitro, and this effect can be inhibited when pR is present.

### Example 7

### KLK Abrogates Cpg-ODN-Induced Production of Pro-Inflammatory Cytokines by Murine Bone Marrow-Derived Dendritic Cells In Vitro

Lipopolysaccharide (LPS) Lipopolysaccharide from Escherichia coli; serotype 0111:B4 (SIGMA Chemicals)

Dose: 100 ng/ml

KLK KLKLLLLLKLK

Dose: 16, 8 μg/ml

CpG-ODN 1668 phosphothioate-modified oligode oxynucleotides containing a CpGmotif: tcc atg acg ttc ctg atg ct, synthesized by NAPS Göttingen GmbH.

Dose: 0.5 nmol/ml

Experimental Groups

1. culture medium

2. LPS

3. KLK

4. CpG-ODN 1668

5. KLK+CPG-ODN 1668

Murine dendritic cells (DC) were generated from bone-marrow precursor cells of C57B1/6 mice as described in example 5. CD11c⁺ sorted BM-DC were incubated either with KLK (16, 8 μg/ml), CPG-ODN 1668 (0.5 nmol/ml) or the combination of poly-L-arginine and CPG-ODN 1668, and, for control purposes, with medium or LPS. Supernatants were harvested after 24 h and analysed for the production of TNF-a and IL-6 using specific ELISAs.

This experiment revealed that CpG-ODN 1668 induces the production of TNF-a and IL-6 by murine BM-DC (FIG. 7). When murine BM-DC were stimulated with KLK alone, we could not detect any of the mentioned pro-inflammatory cytokines in DC-derived supernatants. The CpG-ODN 0.1668-induced production of TNF-a and IL-6 was inhibited when BM-DCs were stimulated with a mixture of CpG-ODN 1668 and KLK.

### Example 8

### The Co-Injection of Poly-L-Arginine Prevents the BCG/CpG-ODN-Induced Systemic Production of TNF-a and IL-6 In Vivo

Mice C57Bl/6 (Harlan/Olac)

Vaccine BCG. (Bacille Calmette Guérin) Vaccine “Pasteur Mérieux”; live vaccine, containing an attenuated strain of Mycobacterium bovis; (Pasteur Mérieux Connaught Austria)

Dose: 5×10⁵ cells of BCG/mouse

Poly-L-arginine 60 (pR60) Poly-L-arginine with an average degree of polymerization of 60 arginine residues; SIGMA Chemicals

Dose: 100 μg/mouse

CpG-ODN 1668 phosphothioate-modified oligodeoxynucleotides containing a CpG motif: TCC ATG ACG TTC CTG ATG CT, synthesized by NAPS GmbH, Göttingen.

Dose: 5 nmol/mouse

Experimental Groups: 4 Mice Per Group

1. BCG

2. CpG 1668

3. BCG+pR60

4. BCG+CpG 1668

5. BCG+CpG 1668+pR60

Mice were injected subcutaneously into the flank with a total volume of 100 μl, containing the above mentioned compounds. One hour after injection blood was taken from the tail-vein and serum was prepared. The amount of the pro-inflammatory cytokines TNF-a and IL-6 in the sera was determined by cytokine-specific ELISAs according to the manufacturer's instructions (R&D Systems, Inc., Minneapolis, Minn.).

This experiment shows that injection of BCG alone or in combination with poly-L-arginine does not induce the production of significant amounts of TNF-a or IL-6 (FIG. 8). In contrast, the injection of CpG-ODN 1668 alone or in combination with BCG induces the systemic production of TNF-a and IL-6. When the BCG vaccine and CpG-ODN were co-injected with poly-L-arginine, the CpG-ODN-induced production of pro-inflammatory cytokines was inhibited.

### Example 9

### Poly-L-Arginine Decrease LPS-Induced Production of Pro-Inflammatory Cytokines by Murine Bone-Marrow-Derived Dendritic Cells In Vitro

Lipopblysaccharide (LPS) Lipopolysaccharide from Escherichia coli; serotype 055:B5 (SIGMA Chemicals)

Dose: 10 ng/ml

Poly-L-arginine 60 (pR60) Poly-L-arginine with an average degree of polymerization of 60 arginine residues; SIGMA Chemicals, P-4663, Lot 68H5903

Dose: 10 μg/ml

Experimental Groups

1. culture medium 12 h/culture medium 12 h

2. culture medium 12 h/pR60 12 h

3. culture medium 12 h/LPS 12 h

4. culture medium 12 h/pR60+LPS 12 h

5. pR60 12 h/LPS 12 h

Murine dendritic cells (DC) were generated from bone-marrow precursor cells of C57Bl/6 mice as described in example 5.

For in vitro-stimulations, day 7-cultured bone marrow cells were incubated for 12 h with the indicated compounds. After centrifugation, supernatant was removed, cells were washed once with medium, then fresh medium with the indicated compounds was added, and the cells were incubated for additional 12 h. Supernatants were harvested and analyzed for the production of TNF-a and IL-6 using specific ELISAs.

This experiment revealed that LPS induces the production of TNF-a and IL-6 by murine BM-DC (FIG. 9). When murine BM-DC were stimulated with poly-L-arginine alone, we could not detect any of the mentioned pro-inflammatory cytokines in DC-derived supernatants. The LPS-induced production of TNF-a and IL-6 was inhibited when BM-DCs were stimulated with a mixture of LPS/poly-L-arginine or when the cells were pre-incubated with poly-L-arginine.

### Example 10

### Poly-L-Arginine, KLK and Poly-D-Lysine Decrease LPS-Induced Production of Pro-Inflammatory Cytokines by Human Dendritic Cells In Vitro

Lipopolysaccharide (LPS) Lipopolysaccharide from Escherichia coli; serotype 055:B5 (SIGMA Chemicals)

Dose: 5 ng/ml

Poly-L-arginine 60 (pR60) Poly-L-arginine with an average degree of polymerization of 60 arginine residues; SIGMA Chemicals, P-4663, Lot 68H5903

Dose: 10 μg/ml

KLK KLKLLLLLKLK

Dose: 16, 8 μg/ml

Poly-D-lysine (pK) Poly-D-lysine Hydrobromide, Sigma P-6403, Lot 108H5909,

Dose: 10 μg/ml

Experimental Groups

1. culture medium 12 h/LPS 12 h

2. culture medium 12 h/pR60 12 h

3. culture medium 12 h/pR60+LPS 12 h

4. pR60 12 h/LPS 12 h

5. culture medium 12 h/KLK 12 h

6. culture medium 12 h/KLK+LPS 12 h

7. KLK 12 h/LPS 12 h

8. culture medium 12 h/pK 12 h

9. culture medium 12 h/pK+LPS 12 h

10. pK 12 h/LPS 12 h

Human dendritic cells (DC) were generated as described in example 6. For in vitro-stimulations, day 7-cultured dendritic cells were incubated for 12 h with the indicated compounds. After centrifugation, supernatant was removed, cells were washed once with medium, then fresh medium with the indicated compounds was added, and the cells were incubated for additional 12 h. Supernatants were harvested and analyzed for the production of TNF-a and IL-6 using specific ELISAs.

This experiment revealed that LPS induces the production of TNF-a and IL-6 by human dendritic cells (FIG. 10). When human DC were stimulated with poly-L-arginine, KLK or poly-D-lysine alone, we could not detect any of the mentioned pro-inflammatory cytokines in DC-derived supernatants. The LPS-induced production of TNF-a and IL-6 was inhibited when DCs were stimulated with a mixture of LPS/poly-L-arginine, KLK or poly-D-lysine or when the cells were pre-incubated with poly-L-arginine, KLK or poly-D-lysine.

### REFERENCES

Andreu, D., and Rivas, L. (1998). Animal antimicrobial peptides: an overview. Biopolymers 47, 415-433.

Ganz, T. (1999). Defensins and host defense [comment]. Science 286, 420-421.

Ganz, T., and Lehrer., R. I. (1999). Antibiotic peptides from higher eukaryotes: biology and applications. Mol Med Today 5, 292-297.

Goldman, R., Bar-Shavit, Z. (1983). On the mechanism of the augmentation of the phagocytic capability of phagocyctic cells by Tuftsin, substance P, neurotensin, and kentsin and the interrelationship between their receptors. Ann N Y Aca. Sci. 419:143-55.

Inaba et al. (1992). Generation of large numbers of dendritic cells from mouse bone marrow cultures supplemented with granulocyte/macrophage colony-stimulating factor. J. Exp. Med. 176:1693

Hancock, R. E. (1999). Host defence (cationic) peptides: what is their future clinical potential? Drugs 57, 469-473.

Karima, R., Matsumoto, S., Higashi, H., Matsushima, K. (1999). The molecular pathogenesis of endotoxic shock and organ failure. Molecular Medicine Today, March 1999, 123-132

Lipford, G. B., T. Sparwasser, M. Bauer, S. Zimmermann, E. Koch, K. Heeg, H. Wagner. 1997. Immunostimulatory DNA: sequence-dependent production of potentially harmful or useful cytokines. Eur. J. Immunol. 27:3420

Sparwasser, T. T. Miethke, G. Lipford, A. Erdmann. H. Häcker, K. Heeg, H. Wagner. 1997. Macrophages sense pathogens via DNA motifs: induction of tumor necrosis factor-a-mediated shock. Eur J. Immunol. 27:1671

Verdijk, R. M., T. Mutis, B. Esendam, J. Kamp, C. J. Melief, A. Brand, E. Goulmy. 1999. Polyriboinosinic polyribocytidylic acid (poly(I:C)) induces stable maturation of functionally active human dendritic cells. J Immunol. 163:57

1. Use of a polycationic compound for the preparation of a medicament for treating or preventing inflammation.

2. Use of a polycationic compound for the preparation of a medicament with reduced inflammatory potential.

3. Use according to claim 1 or 2, characterized in that said medicament is a vaccine.

4. Use according to any one of claims 1 to 3, characterized in that said medicament comprises an antigen.

5. Use according to claim 4, characterized in that said antigen is selected from the group consisting of an antigen from a viral or a bacterial pathogen, an antigen from an eukaryotic pathogen, a tumor antigen, an autoimmune antigen or mixtures thereof.

6. Use according to any one of claims 1 to 5, characterized in that said polycationic compound is a polycationic peptide, preferably a basic polypeptide, an organic polycation comprising peptide bonds or mixtures thereof.

7. Use according to any one of claims 1 to 6, characterized in that said polycationic compound is polylysine, polyarginine, a polypeptide containing more than 50% of basic amino acid residues in a range of more than 5, especially more than 10, amino acid residues or mixtures thereof.

8. Use according to any one of claims 1 to 7, characterized in that said medicament further comprises a compound with an inflammatory potential.

9. Use according to any one of claims 1 to 8, characterized in that said medicament is used in combination with a medicament with an inflammatory potential.

10. Use according to any one of claims 1 to 9, characterized in that said medicament further comprises immunogenic nucleic acid molecules.

11. Use according to any one of claims 1 to 10, characterized in that said medicament further comprises immunogenic oligodesoxy nucleic acid molecules (ODNs), especially ODNs comprising CpG motifs (CpG-ODNs), inosine containing ODNs (1-ODNs) or mixtures or combinations thereof.

12. Use according to any one of claims 1 to 11, characterized in that said medicament is a locally acting medicament.

13. Use according to any one of claims 1 to 12, characterized in that said medicament further comprises an active substance, said active substance having an affinity to said polycationic compound.
